(19) United States
(12) Patent Application Publication
(10) Pub. No.: US 2023/0100365 A1
(43) Pub. Date: Mar. 30, 2023

(54) Automatic classification of healthy and disease conditions from images or digital standard 12-lead ECGs

(71) Applicant: Technion Research and Development Foundation Ltd., Haifa (IL)

(72) Inventors: Yael Yaniv Schuster, Haifa (IL); Assaf Schuster, Haifa (IL); Vadim Gliner, Haifa (IL)

(73) Assignee: Technion Research and Development Foundation Ltd., Haifa (IL)

(21) Appl. No.: 17/936,378

(22) Filed: Sep. 28, 2022

Related U.S. Application Data
(60) Provisional application No. 63/261,835, filed on Sep. 29, 2021.

(51) Int. Cl. G16H 50/20; G06T 7/00; A61B 5/349

(52) U.S. Cl. CPC G16H 50/20; G06T 7/0012; A61B 5/349; G06T 2207/30048; G06T 2207/20081

(57) ABSTRACT

A method for automatic determining of a state of a heart of a patient based on multiple-lead ECG information of the patient, the method includes (a) receiving the multiple-lead ECG information of the patient, by a computerized system; and (b) applying one or more machine learning processes on the multiple-lead ECG information of the patient to determine the state of a heart of the patient, wherein the state of the heart comprises at least one heart condition of multiple types of heart conditions. One, some or all of the one or more machine learning processes was trained using a dataset that comprises multiple computer generated images, the multiple computer generated images represent images acquired by an image acquisition process of ECG plots, wherein the ECG plots are generated based on digital multiple-lead ECG signals.

### CROSS REFERENCE

This application claims priority from U.S. provisional patent Ser. No. 63/261,835 filing date Sep. 29, 2021 which is incorporated herein by reference in its entirety.

### BACKGROUND

Standard 12-lead ECG is used as a primary clinical tool to diagnose changes in heart conditions because of its simplicity, noninvasive character and low cost effective. This type of ECG (2.5 sec of each lead and 10 sec from one lead) was shown as a single adequate source to diagnose cardiac rhythm and morphology such as cardiac arrhythmia, acute and prior myocardial infarctions, pericardial disease, and atrial or ventricular enlargement.

The value of automatic 12-lead ECG diagnostic approaches lies in their ability to screen the general population and to provide a second opinion for care provider. Since 1957, attempts to computerize interpretation of ECG recordings have been made with emphasize on atrial fibrillation (AF). Despite the current technology specifically advanced machine learning and artificial intelligence (AI) methods, the clinical use of the computerized ECG remained limited and a standard 12-lead ECG is read over by cardiologists. There are several challenges that embrace the design of clinically relevant automatic algorithm of classification of cardiac conditions from 12-lead ECG: (i) Accuracy level. Currently automatic algorithms have had mediocre performance. Achieving high accuracy is the first step to clinically test an automated 12-lead ECG classification system. Note that even experienced cardiologists or expert can disagree, thus achieving high accuracy is challenging even for cardiologists. (ii) 12-lead ECG vs. one lead ECG. In the recent years advanced machine learning and AI methods were used to classify one lead ECG. The advance of AI technology is the use of features that are either unknown or cannot be easily visually analyzed. When validated against an independent test dataset annotated by a consensus committee of board-certified practicing cardiologists, the sensitivity of the automatic program for most rhythm classes slightly exceeded that of the average cardiologist. However, single-lead analysis can be used only to classify very few diseases that are mainly rhythm conditions. However, using 12-lead ECG one can detect rhythm and morphology conditions and not only rhythm changes. (iii) 12-lead ECG format. Because standard 12-lead ECG databases are sometimes available in ECG digital signal format and sometimes as machine plot image, a general, hybrid approach that allow disease identification from both must be developed. Moreover, because machine plot images are recently scanned by a mobile device, an approach must take into account artifacts caused by mobile device image acquisition. (iv) It is very common for more than a one disorder to concomitantly present in the same ECG record. However, majority of the automatic classification of cardiac diseases systems are multiclass model (i.e., class the ECG into one category). Only a generic architecture that can be applied to many diseases of different types and detect all disorders appearing in the same record would have a physiological merit. (v) Unknown conditions. AI system must be trained on labeled data, thus includes only a certain set of labeled disorders. However, other disorders than the one considered in the multiclass model do exist, a fact which raises the question whether published results are applicable to these additional diseases. To overcome these limitations a generic architecture that can be extended to additional diseases with relatively minimal effort must be designed. (vi) Standardization of applied algorithms. To apply AI methods, a large dataset is essential. However, to the best of our knowledge, standard, open-source, 12 lead ECG records from many patients and healthy volunteers does not exist.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1D are examples of data formats;

FIGS. 2A and 2B illustrate CNN architectures;

FIGS. 3A-3I illustrate the receiver operation characteristic (ROC) of each disease classification;

FIG. 4 illustrates the receiver operation characteristic (ROC) of atrial fibrillation detection;

FIG. 5 illustrates the accuracy of binary classification of atrial fibrillation as a function of training set size;

FIG. 6 illustrates an example of an infrastructure;

FIG. 7 illustrates an example of front end user interface layout;

FIG. 8 illustrates an example of screens 90 displayed by an application;

FIG. 9 illustrates an example of a method;

FIG. 10 illustrates an example of a method; and

FIG. 11 illustrates examples of systems.

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using electronic components and circuits known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that can be executed by the system and should be applied mutatis mutandis to a non-transitory computer readable medium that stores instructions that once executed by a computer result in the execution of the method.

The terms “digital multiple-lead ECG information”, “digital multiple-lead ECG signals”, “ECG digital signals”, and “digital 12-lead ECG signal data” are used in an interchangeable manner and are sued to describe a digital representation of ECG signals acquired from a multiple lead ECG unit. The digital multiple-lead ECG signals differ from an ECG plot that is plotted on a background of an ECG paper.

There is provided a general, hybrid approach to a standard 12-lead ECG database, that overcomes all the above challenges and would classify to normal sinus or one or more of the following disorder types: atrial fibrillation (AF), first-degree atrioventricular block (I-AVB), left bundle brunch block (LBBB), right bundle brunch block (RBBB), premature atrial contraction (PAC), premature ventricular contraction (PVC), ST-segment depression (STD) or ST-segment elevation (STE).

Data Source and Standard 12-Lead ECG Database Generation

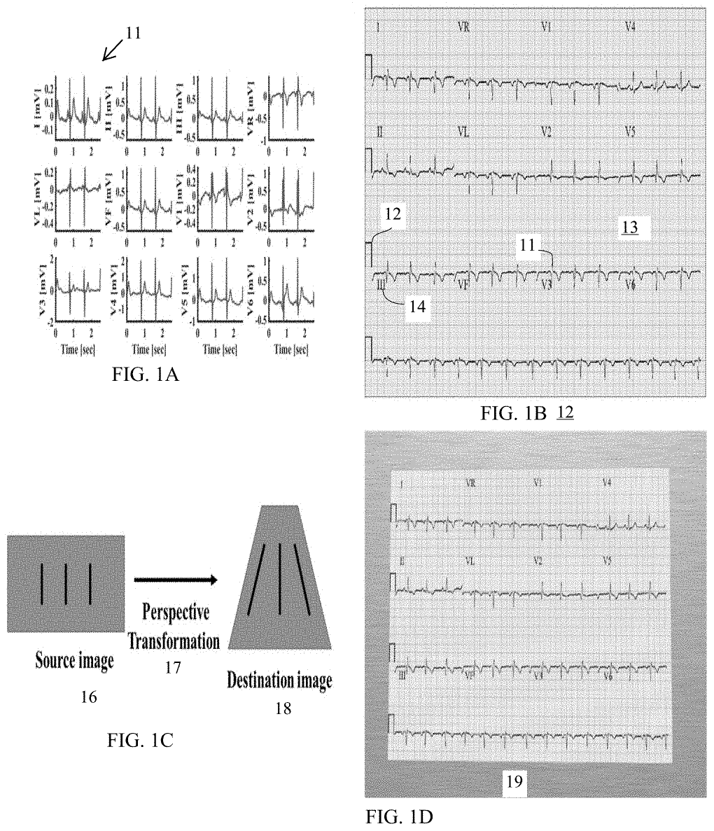

FIGS. 1A-1D are example of data formats. FIG. 1A illustrates digital 12-lead ECG signal data 11. FIG. 1B illustrates Rendered ECG image 12. The ECG digital signals 11 was plotted on a publicly available ECG paper template 13. The lead labels 11 indicate lead recording location on the template (2.5 sec each). The lower long lead recording is lead II (10 sec). The calibration signal 12 is also drawn on the left side of each row. FIG. 1C illustrates an image 16 without perspective transformation (left side of the image) and an image 18 with (right side of the image) perspective transformation. A perspective transformation 17 imitates human (or camera) view of a rectangular ECG plot. Other transformations and/or image amendments may be used to mimic an image acquisition process of the ECG plot. FIG. 1D illustrates a computer generated image such as a rendered image after applying a random perspective transformation and a random background (or any selected background) to imitate acquisition by a mobile device, such as a smartphone.

To construct our database, we used 6,877 published patient records (female 3,178; male 3,699), of durations of 6-60 sec, that were collected in 11 hospitals. ECG recordings were acquired at a sampling rate of 500 Hz. The data were classified by board-certified practicing cardiologists in these 11 hospitals as normal sinus or one or more of the following conditions: AF, I-AVB, LBBB, RBBB, PAC, PVC, STD or STE. This database was created from standard 12-lead ECG records (either ECG digital signal (FIG. 1A) or image (FIG. 1B)) consisting of 2.5-sec recording for each one of 12 leads and 10-sec recording for lead II. Recordings longer than 10 seconds were divided to 10-sec segments with no overlaps. Each 10-sec segment was used to generate four standard ECG records by taking 2.5 sec of 12 leads without overlap, and the same 10 sec of lead II. 11 ECG records shorter than 10 sec were omitted.

A database of 41,830 recordings (in hdf5 format compatible with PyTorch) from 6,866 patients (female 3,174; male 3,692) was created. The dataset scheme follows that of other known datasets (MNIST for example). A “one-hot” classification binary vector with a length of 9 was generated for each database entry: (1) Normal, (2) AF, (3) I-AVB, (4) LBBB, (5) RBBB, (6) PAC, (7) PVC, (8) STD and (9) STE. The corresponding vector entry was “1” if the disease existed in the database entry and otherwise was “0”. Table 1 presents the number of patients and records in each category.

Table 1 illustrates An example of a classification of source data, with number of patients and records for each condition type. Atrial fibrillation (AF), first-degree atrioventricular block (I-AVB), left bundle brunch block (LBBB), right bundle brunch block (RBBB), premature atrial contraction (PAC), premature ventricular contraction (PVC), ST-segment depression (STD) and ST-segment elevation (STE).

TABLE 1

| Condition type | # of patients | # of records |
|---|---|---|
| Normal | 918 | 5453 |
| AF | 1220 | 7080 |
| I-AVB | 721 | 3969 |
| LBBB | 235 | 1356 |
| RBBB | 1853 | 10401 |
| PAC | 614 | 4552 |
| PVC | 699 | 5598 |
| STD | 868 | 5069 |
| STE | 220 | 1458 |

The Image Dataset

ECG recordings are available in two modalities—ECG digital signal or paper plots. Our goal was to show that automatic identification of diseases can be applied on standard available ECG data, regardless of their format. Because there is no accessible dataset of labeled ECG plots with a sufficiently large volume of data, we generated such data from the ECG digital signal dataset.

A publicly available template of ECG paper was used to generate ECG images (see FIG. 1B). Of note, the machine learning techniques we used were agnostic to the specific ECG paper template that is used; identical results can be obtained using any template. Each small red box represents 0.04 sec (imitating ECG paper that is fed into the machine at a rate of 25 mm/sec) and one-millimeter height. ECG lead label was added next to each lead plot and a calibrated signal (10 mm-high and 0.2 sec-wide) was added at the beginning of each ECG line.

When the data are available in paper plot form only, the easiest way to make it accessible to an automatic classification system is to capture the plot image, which then serves as the input to the system. Today, common practice is to capture the plot image using cameras in smartphones or other mobile devices. However, this practice is likely to incorporate distortions in the resulting image. Because of lack of an accessible database comprising labeled ECG images captured by smartphones, we aimed to imitate the artifacts caused by this acquisition method. Specifically, we applied random perspective image transformations to imitate projection from 3D view into 2D image (FIG. 1C) and added random backgrounds resembling various table textures (FIG. 1D).

Data Sets for Developing and Testing the Network

After an initial split of the data into the development (83% of the population-36,000 records) and holdout (17% of the population-5,830 records) data sets, the development data set was further divided into training (95% of the development set) and internal validation (5%) data sets. To avoid cross-contamination, the development and holdout data sets consisted of different sets of patients. Similarly, the training and test sets consisted of different sets of patients. Training both the deep convolution neural network (CNN) for digital data (CNN-dig) and the CNN for plot images (CNN-ima) networks, was done using the Adam optimizer with binary cross-entropy as the loss function. The internal validation data set was used to tune the hyper-parameters and to select the optimal model. The same data split (training, validation, and testing sets) was used for both the ECG digital signal and image networks.

Overview of the Deep Network Model

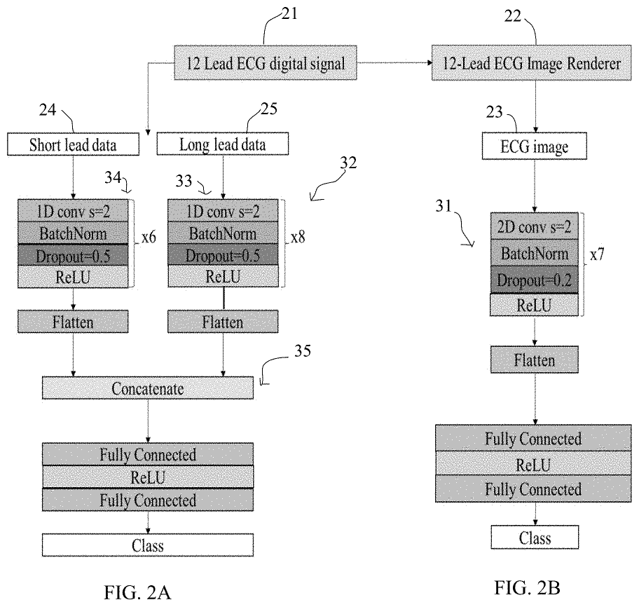

FIGS. 2A and 2B illustrate CNN architectures.

FIG. 2A illustrates a 12-lead ECG digital signal data classification net CNN-dig 32, and FIG. 2B illustrates a 12-lead ECG image classification net CNN-ima 31.

CNN-ima 31 may be fed by computer generated images such as rendered ECC image 23. CNN-dig 32 may be fed by ECG digital signals such as 12 lead ECG digital signal 21. In FIG. 2B the 12 lead ECG digital signal 21 is converted by 12-lead ECG image rendered to rendered image 22.

The 12 lead ECG digital signal 21 includes long lead data 25 and short lead data 24.

The long lead data 25 is fed to a long lead branch 33 of CNN-dig 32 and the short lead data 24 is fed to a short lead branch 34 of CNN-dig. Each of the branches may include a convolution layer, a batch normalization layer a dropout layer and an ReLU layer. The outputs of the ReLU layers of the two branches may be merged—for example may be concatenated and fed to a shared portion 35 of CNN-dig.

In both networks, the input enters a convolution layer with a stride step of 2. Next in line is a batch normalization layer in which the batch distribution is normalized. The dropout layer randomly deletes a fraction of the network edges with a given probability during the training to improve robustness. Next, the 3D convolution output is flattened, goes through a fully connected classification layer and is thresholder for the final outcome.

There may be multiple pairs of CNN-dig and CNN-image. And each pair may be trained on a single disease and generates a binary output for each input: in class or out of class.

CNNs were implemented using the Pytorch Framework with Python. The same basic architecture was used in different variations for all ECG digital signal identification networks CNN-dig and for all image identification networks CNN-ima. While CNNs are mainly applied to images, we adjusted the network architecture and the convolution kernel size to produce spatial and temporal feature extraction layers. The network was trained by adjusting the weights of the convolutional filters to extract meaningful and relevant features in an unsupervised way.

Both the ECG image and ECG digital signal networks were built using stacked blocks of convolutional, batch normalization and dropout. For training with ECG digital signal data, two separate stacked blocks were used to extract temporal features from short leads and from the long lead. After flattening each stacked block (either short or long lead), the extracted features from both blocks were concatenated and were used as input to a fully connected network. Both networks used a linear rectifier (ReLU) as their activation function.

The difference between the image CNN-dig and ECG digital signal CNN-ima networks was in the convolution output. The image network is one stack of convolutional layers followed by a fully connected layer.

Another difference is the 1D convolution kernels in the ECG digital signal network (17) vs. 2D convolution kernels in the image network (7×7). Hyperparameters (batch size, initial learning rate, number of nodes in the fully connected layers, and number of convolutional layers) were adjusted during training to obtain the optimal model. Initial learning rates were set in the range 1e-2 to 1e-5, and testing batch sizes varied in the range of 50-150 for the image network CNN-ima, and 150-256 for the ECG digital signal network CNN-dig. Selected hyperparameters included batch size of 256 for the ECG digital signal network, whereas for the image network we chose batch size of 150. Note that for the ECG digital signal case, the same net architecture was used for each condition (8 diseases and normal sinus), while, obviously, the model parameters (the outcome of the training process) differed.

Model Evaluations and Statistical Methods

For each disease, a binary classifier (disease exists or not) was designed with an output P, where P was in the range [0-1]. In cases of inconclusive discrimination, P will be closer to 0.5, thus, a trivial threshold of 0.5 was used for final binary classification. In the training stage, the networks were fed the entire training set. After every iteration (epoch), the model was evaluated on the validation set. The software callback saved only the best model based on the validation performance (checkpoints). To avoid overfitting, we used the early stopping method. Training was terminated when the validation performance ceased to improve over 5 or 10 consecutive epochs, for CNN-ima or CNN-dig, respectively. Our experience showed that CNN-dig had to be trained for 100 epochs, whereas the CNN-ima achieved optimal results already after 6 epochs.

The following metrics were used to assess performance of the networks:

$$a . TP = \frac{\# correctly detected disease occurences}{\# episodes with presence of the disease} \\ b . TN = \frac{\# correctly detected disease absence}{\# episodes with absence of the disease} \\ c . FN = \frac{\# misdetected disease occurences}{\# episodes with presence of the disease} \\ d . FP = \frac{\# incorectly classified episodes}{\# episodes with absence of the disease} \\ e . Sensitivity : True positive rate ( TPR ) = \frac{TP}{TP + FN} \\ f . Specificity : True negative rate ( TNR ) = \frac{TN}{TN + FP} \\ g . Precision or positive predictive value : PPV = \frac{TP}{TP + FP} \\ h . Negative predictive value : NPV = \frac{TN}{TN + FN} \\ i . Accuracy : ACC = \frac{TP + TN}{TP + TN + FP + FN} \\ j . {F}_{1} = \frac{2 * TP}{2 * TP + FP + FN}$$

Results

Creation of relatively large, publicly available, 12-lead ECG standard database

A total of 6,866 patients and volunteers and 41,830 standard 12-lead ECG records were created. Each digital ECG recording was classified to one or more of nine categories (see Methods section). In total, there were 13.03% normal, 16.92% AF, 9.48% I-AVB, 3.24% LBBB, 24.86% RBBB, 10.88% PAC, 13.38% PVC, 12.11% STD and 3.48% STE recordings. The training set, including the validation set, was comprised of 36,000 ECG recordings. The testing set was comprised of 5,830 ECG recordings collected from individuals that were not included in the training set.

Generic, Optimal Convolution Kernel Size for ECG Digital Signal Data

Beyond achieving high classification accuracy for eight disease classes and normal sinus, the ultimate goal of this work was to identify a network architecture suitable for as many other diseases as possible, including both morphological and rhythm disorders. Success in doing so will make our solution general and applicable to many additional disease types with very small engineering effort. To this end, we searched for the optimal convolution kernel size providing high accuracy for all conditions. Small convolution kernels are expected to be more suitable for identifying morphological disorders, while large convolution kernels are expected to be sensitive to changes between subsequent beats and therefore suitable for identifying both rhythm and morphological disorders. Table 2 shows that a kernel size of 17 provided the best average performance over all types.

Table 2 illustrates an example of a convolution kernel size optimization. The results in the table are for testing on the validation set (1000 records). Atrial fibrillation (AF), first-degree atrioventricular block (I-AVB), left bundle brunch block (LBBB), right bundle brunch block (RBBB), premature atrial contraction (PAC), premature ventricular contraction (PVC), ST-segment depression (STD) and ST-segment elevation (STE).

TABLE 2

| | Convolution kernel size/Arrhythmia type | | | | | | | | |
|---|---|---|---|---|---|---|---|---|---|
| | 3 | 5 | 7 | 9 | 11 | 13 | 15 | 17 | 19 |
| Normal | 95.1 | 95 | 96.2 | 96.5 | 96.5 | 97 | 97 | 96.7 | 97.2 |
| AF | 96.7 | 94.7 | 98 | 97.8 | 98 | 98.1 | 98.2 | 98.1 | 98.2 |
| I-AVB | 97.4 | 98.6 | 98.2 | 97.7 | 97.6 | 97.8 | 98.8 | 98.7 | 98.7 |
| LBBB | 99.9 | 100 | 100 | 100 | 100 | 100 | 100 | 100 | 100 |
| RBBB | 98.5 | 99.2 | 99.2 | 98.9 | 98.8 | 99.1 | 99.5 | 98.6 | 98.8 |
| PAC | 90.9 | 89.6 | 91.5 | 92.3 | 91.7 | 92.3 | 93.7 | 94.1 | 93.2 |
| PVC | 89.2 | 89.2 | 89.4 | 89.7 | 88.8 | 91.6 | 91.5 | 92.9 | 92.3 |
| STD | 87 | 90.9 | 91.2 | 91.3 | 91 | 92 | 90.9 | 91.2 | 91.3 |
| STE | 99.9 | 98.4 | 98.4 | 100 | 98.8 | 98.3 | 98.3 | 98.4 | 98.3 |
| Mean | 95.0 | 95.0 | 95.8 | 96.0 | 95.7 | 96.2 | 96.4 | 96.5 | 96.4 |

Detection of multiple disorders from ECG digital signal data: generality and extensibility

Analysis of 12-lead ECG recordings must be sufficiently sensitive to identify and differentiate between concomitant conditions. Assignment of such a task to a multiclass classifier, would require training for the theoretical number of disease combinations (2\hat{ }8), making our classifier cumbersome, inefficient, and practically impossible to train due to lack of data. The same logic makes the multiclass classifier approach non-scalable for identifying even larger sets of diseases.

For this reason, we took a different approach, which used a generic architecture that is separately trained for binary identification of each disease. Using our approach, given a set of N trained models and for any m<N, it takes N inference operations to detect a set of m existing disorders in a single ECG recording. Such network training is much more efficient, the results are more accurate, and, most importantly, requires a reasonable number of samples. Furthermore, with this approach, the effort required to extend the system to detect an additional disease is relatively low, simply requiring training of yet another model using the same architecture.

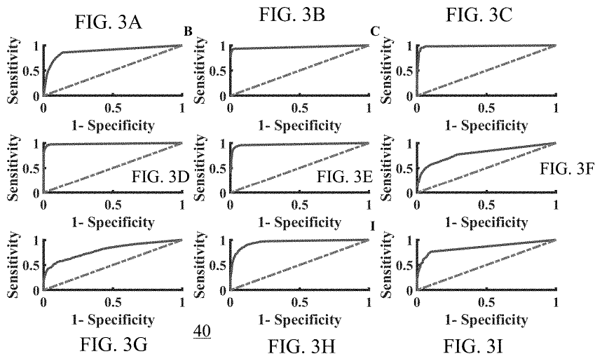

FIGS. 3A-3H includes curves (collectively denoted 40) that illustrate the receiver operation characteristic (ROC) of each disease classification. Overall area under the curve for each disease was between 0.8-0.98.

These figures illustrate receiver operation characteristic (ROC) of disease identification from ECG digital signals, using CNN-dig.

FIG. 3A illustrates an example of a normal heart condition, the area under curve is 0.89.

FIG. 3B illustrates an example of a AF, the area under curve is 0.96.

FIG. 3C illustrates an example of a I-AVB, the area under curve is 0.98.

FIG. 3D illustrates an example of a LBBB, the area under curve is 0.98.

FIG. 3E illustrates an example of a RBBB, the area under curve is 0.97.

FIG. 3F illustrates an example of a PAC, the area under curve is 0.8.

FIG. 3G illustrates an example of a PVC, the area under curve is 0.8.

FIG. 3H illustrates an example of a STD, the area under curve is 0.95.

FIG. 3I illustrates an example of a STE, the area under curve is 0.85.

Table 3 shows the accuracy for each category, which was 92.9-100%, depending on the disease type. The identification accuracy for each disease type, based on a ECG digital signal. Atrial fibrillation (AF), first-degree atrioventricular block (I-AVB), left bundle brunch block (LBBB), right bundle brunch block (RBBB), premature atrial contraction (PAC), premature ventricular contraction (PVC), ST-segment depression (STD) and ST-segment elevation (STE).

TABLE 3

| Disease type | Accuracy |
|---|---|
| Normal | 0.96 |
| AF | 0.98 |
| I-AVB | 0.99 |
| LBBB | 1.00 |
| RBBB | 0.99 |
| PAC | 0.92 |
| PVC | 0.92 |
| STD | 0.90 |
| STE | 0.98 |

To identify trends in our network misidentification, we generated a confusion matrix (FIG. S1). Because our method is based on independent identification of each cardiac disorder, only data from the test set which included only one classified disease (annotated as well as predicted) were used for the confusion matrix. Out of 5830 records, 3184 were used to generate the matrix. Two significant trends of misidentification were detected—PAC was mistakenly classified as either RBBB or STD and PVC was mistakenly classified as STD.

Atrial Fibrillation Identification from ECG Images

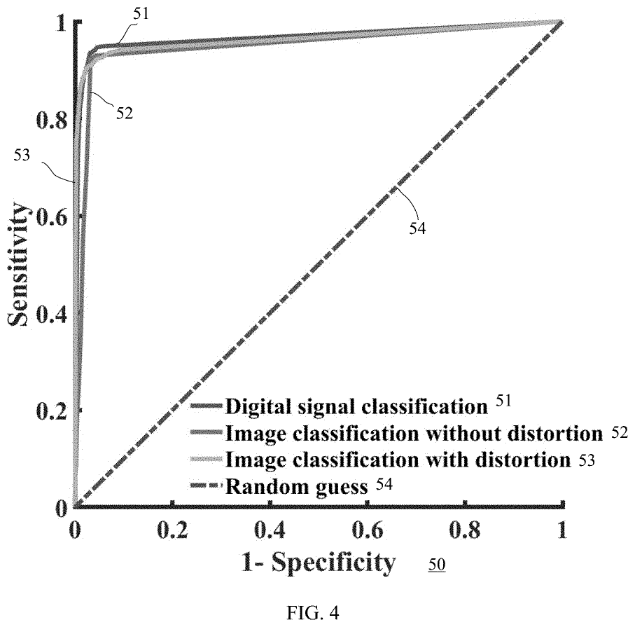

Because of added redundant information (e.g., red squares of ECG plot), it may seem that identifying diseases from 2D images is harder than doing so with 1D ECG digital signals. We challenged this intuition by training CNN-ima to identify AF, the most common arrhythmia, using images only. FIG. 4 illustrates the ROC curve for digital vs. image input signals. Similar degrees of accuracy were achieved with digital (98%) as compared to images (96%) input signals.

FIG. 4 illustrates the receiver operation characteristic (ROC) of atrial fibrillation detection. ROCs of ECG digital signal classification 51 (area under curve: 0.96), ECG image classification without distortions 52 (area under curve: 0.94), ECG image classification with distortions 53 (area under curve: 0.96) and random guess 54 (area under curve: 0.5).

AF Identification from ECG Images with Mobile Device Acquisition Distortion

In the modern age, images of ECG plots, needed for automatic image classification, are likely to be acquired using smartphones, and therefore are likely to be distorted. For lack of adequate labelled dataset, distorted image ECG plots were simulated by employing random perspective transformations as well as incorporating random backgrounds. FIG. 4 illustrates the ROC curve for digital versus image signals, with versus without perspective transformation and random texture background. As previously shown, ECG image identification was slightly less accurate than that of ECG digital signal identification. In addition, identification of ECG images with distortion was a bit less accurate than without.

Classification Accuracy vs. Amount of Data

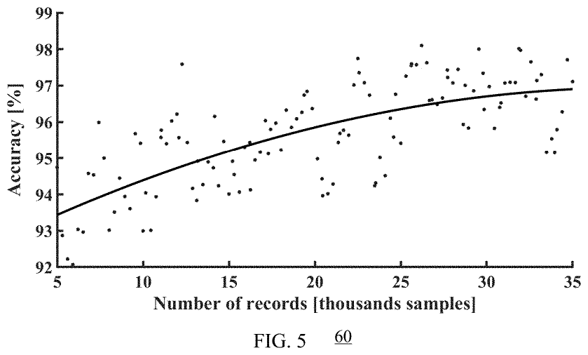

In deep learning, the output accuracy depends on the number training samples. To determine the size-effect of our results and to quantify how additional data may improve them, we explored the dependence of AF accuracy on the number of training samples. To this end, the CNN-dig model was trained with increasing amounts of training data, and then tested each time with the same test set. FIG. 5 includes graph 60 that shows how increasing the volume of training data increased the accuracy of the results. Moreover, the fitting curve (y=a·sin (b·x+c)), coefficients which were found by optimization) showed that no saturation was achieved, namely, that additional of further data would result in higher accuracy.

Discussion

Standard 12-lead ECG is one of the most common tools used for cardiac disease diagnosis and is easily accessible at any clinic. This work provided a proof-of-concept that such an automated system can be created and can reach high accuracy using deep learning tools. We proposed a generic deep network architecture that can be applied to many conditions of different types (i.e., either rhythm or morphology conditions) with high accuracy, can detect all disorders appearing in the same record, and can be extended to additional diseases with relatively minimal effort. In addition, it demonstrated that this approach is applicable to both digital ECG and ECG image signals, including images captured by smartphones. Such automated system would provide a second opinion on manually assessed ECG results and cost-effectively scan massive at-risk populations.

Creation of a New Open-Source Database

12-lead ECG is essential for accurate diagnosis of both ECG morphological and rhythm disorders. Moreover, this device can be found at any clinic. However, diagnostics are currently manually performed by clinicians, without any means of automation. AI methods provide promising new platforms for development of such automatic arrhythmia detection tool. To apply these methods, a large dataset was essential. However, to the best of our knowledge, standard, open-source, 12 lead ECG records from many patients and healthy volunteers does not exist. As part of this research, we built such a database, which is accessible upon request and compatible with a well-known and frequently used deep learning framework (PyTorch). We believe that availability of this database will very positively impact future research in the field.

Design of Generic, Extensible Deep Network Architecture for Cardiac Disease Identification

A multiclass model was recently suggested for automatic classification of cardiac diseases. However, the straightforward learning approach proposed in these works had two critical limitations. First, it is very common for more than a one disorder to concomitantly present in the same ECG record. Second, other disorders than the one considered in the multiclass model do exist, a fact which raises the question whether published results are applicable to these additional diseases. To overcome these limitations, we proposed a generic deep network architecture that can be applied to many diseases of different types with high accuracy, can detect all disorders appearing in the same record, and can be extended to additional diseases with relatively minimal effort.

The advantages of this solution were demonstrated by generation of binary classification (exist or not) for the eight most-common rhythm and/or morphological cardiac arrhythmias. The solution was also capable of identifying normal sinus rhythm. Each of the binary classification networks was trained independently, and therefore did not negatively impact other classes. Thus, these datasets comprising other disorders can be easily incorporated with exist training data and used with the same network architecture as developed here, without affecting the high accuracy of our system.

Real-Life Data Modalities: ECG Digital Signals and Printed Plots

The deep network architecture constructed in this work was designed to be compatible with both digital 12-lead signals (i.e., 13 signals) and with standard plots of all leads, which are typically printed on paper. The presented generic architecture reached excellent accuracy for both CNN-ima and CNN-dig networks. Despite the increase in data dimensionality and the incorporation of redundant information, such as background pixels, CNN-ima suffered only a negligible accuracy reduction. Our results show that the network can extract the important information from the images and ignore the redundant parts.

In the age of smartphones and Internet of Things, images of an ECG plot can be acquired using a mobile device. Doing so, however, is prone to background noise and change of plot perspective. The results of this work show that our generic network architecture can deal with such input distortions without significantly affecting output accuracy.

Amount of Data

The neural network approach is subject to inherent limitations, including reliance on large volumes of data. However, neural networks may offer a higher level of accuracy than other statistical methods as they are only dependent on the data and can operate independently of the biases of the investigator. We showed here that increasing the amount of data improved the AF detection accuracy. Moreover, the volume of data used here did not reach system accuracy saturation. Namely, additional data may very well further improve the results. This observation may also applies for the other tested disorders: it is plausible that our (already highly accurate) results will further improve with the availability of additional datasets.

Conclusions

Using AI tools to analyze 12-lead ECG recordings enabled detection of cardiac disorders with high accuracy. The ability of an AI algorithm to deal with both digital and image signals will be of high value at any clinic.

Heart diseases are the number one killer in the Western world. Currently, the most effective method to diagnose heart diseases is to use the 12-lead ECG. However, to analyze the results, an experienced cardiologist is needed. Note, that even experienced cardiologists do not always agree on the ECG poses an additional challenge. Interestingly, although many of these ECG machines provide automatic classification, their results are so poor that the majority of the cardiologists ignore them. Moreover, there is a variety of ECG machines often at the same hospital and there is no one platform that can deal with all. Finally, in the majority of hospitals in the world, the output of the 12-lead ECG is a paper rather than a digital record, and thus an automatic diagnostic system must deal with either digitized or paper plot. ECG is not the only data that is stored in a paper rather than a digital record. For example: fetus monitoring, EEG data etc. The diagnostic accuracy achieved by algorithms is still worse than that of cardiologists for rare cardiac diseases, or on a par for prevalent diseases. The current automatic algorithms cannot retrain themselves and thus improve their outcome. The data is not digitized.

The platform can use as an input either digital data or our application can capture the paper plot by a smart phone camera. The clinician may send either the digital data or the photo with his diagnosis. The application then transmits the image to a service executed by a central server or a cloud. The platform uses classical image processing tools to overcome noise in the ECG digital signal or in the image (shadow etc). The platform analyzes the data by any AI algorithm to classify the specific clinical data and allow physicians to instantly get a second diagnostic opinion and to keep a digitalized record of the physiological signal. Each such consultation will provide the application with another labeled recording that will allow to train the algorithm. Under the hood, as the amount of data available for training the classifier constantly grows, the algorithm will retrain itself periodically. In this way the algorithm will constantly improve its accuracy and confidence, enabling more precise analysis of prevalent diseases as well as better diagnosis of more types of rare diseases

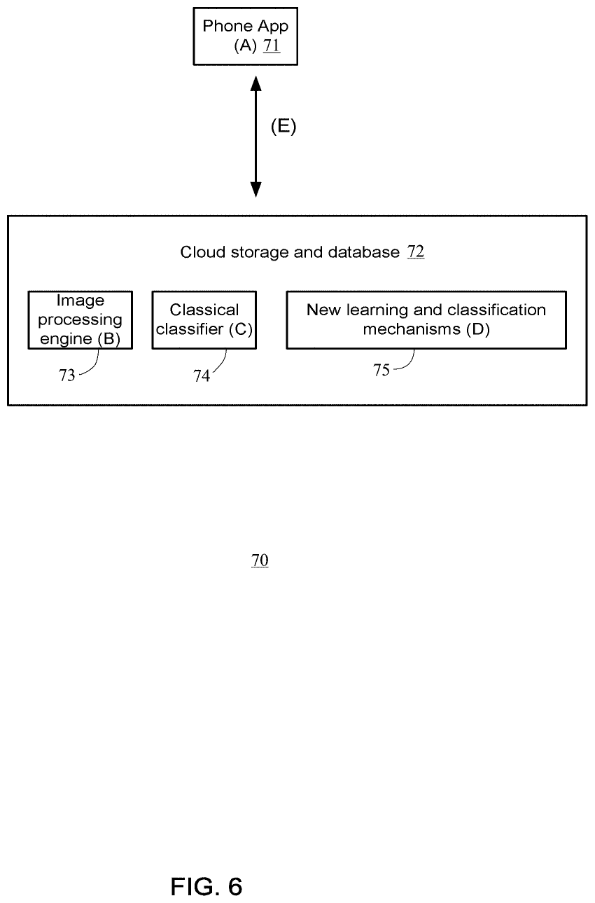

FIG. 6 provides a general system topology. The includes front-end mobile application 71 of a client for acquiring an image of ECG plot, and a cloud storage and database 72 that includes an image processing engine 73 for extracting the 12 lead vectors from a snapshot of a 12-lead ECG, a Classic (e.g., SVM) classifier 74 of 12-lead digitized data and a deep learning classification engine (denoted new learning and classification mechanisms 75). The cloud storage and database 72 may also include a privacy preserving database and storage of both the processing engines and the incoming data.

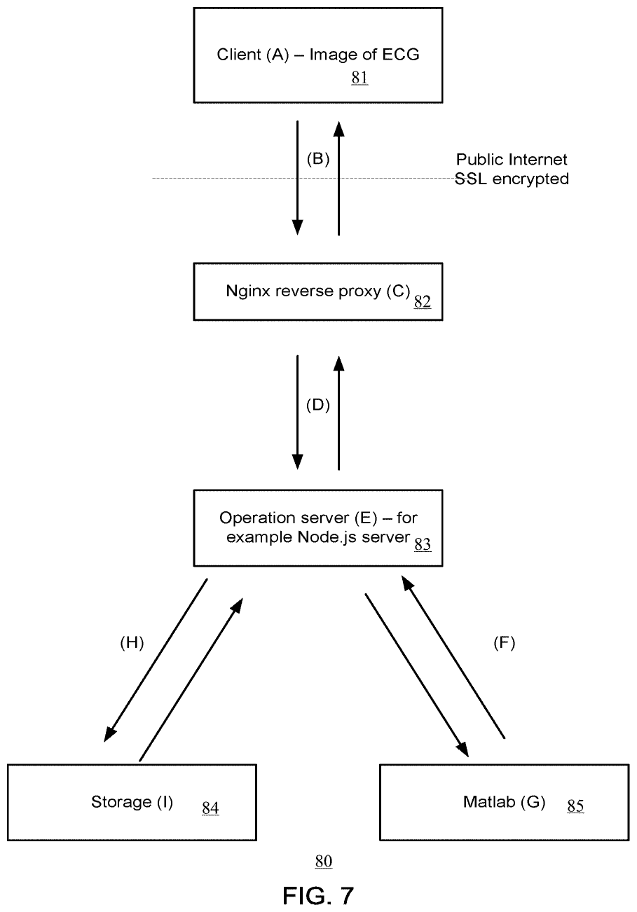

FIG. 7 provides an example of an infrastructure topology 80 that includes: (A) Client side 81: any mobile device with camera and connection to the internet. (B) Communication with the front-end part of the server using public internet (SSL Encrypted). (C) A powerful Nginx reverse proxy server 82 which is responsible for receiving files and other data from the user. (D) HTTP protocol used for communication between the reverse proxy server and the operation server. (E) Operation server 83 which is responsible for routing the data either to processing unit or to output to the user. (F) HTTP protocol used for communication between the operation server and the processing unit. (G) Matlab environment 85 running all the image and signal processing, machine learning and other required algorithms. (H) A process saving received files to storage 84. (I) Server storage comprising all the data ever received by the server. Other protocols may be used and/or other communication devices may be used for transmitting any related information including the acquired ECG image, and/or any other storage and/or processing elements may be used.

This application provides a significant technical improvement over the prior art—especially an improvement in computer science.

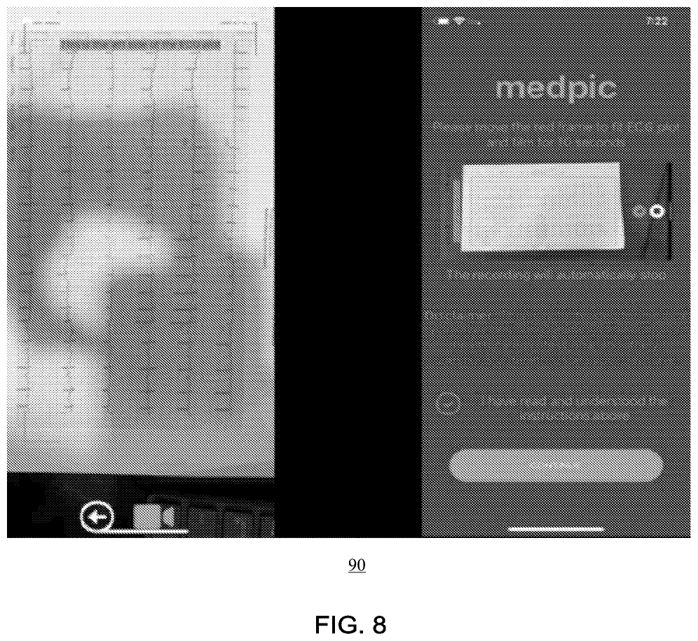

FIG. 8 illustrates an example of screens 90 displayed by an application. The screens include an acquired image of an ECG plot and an instruction bearing screen of the application.

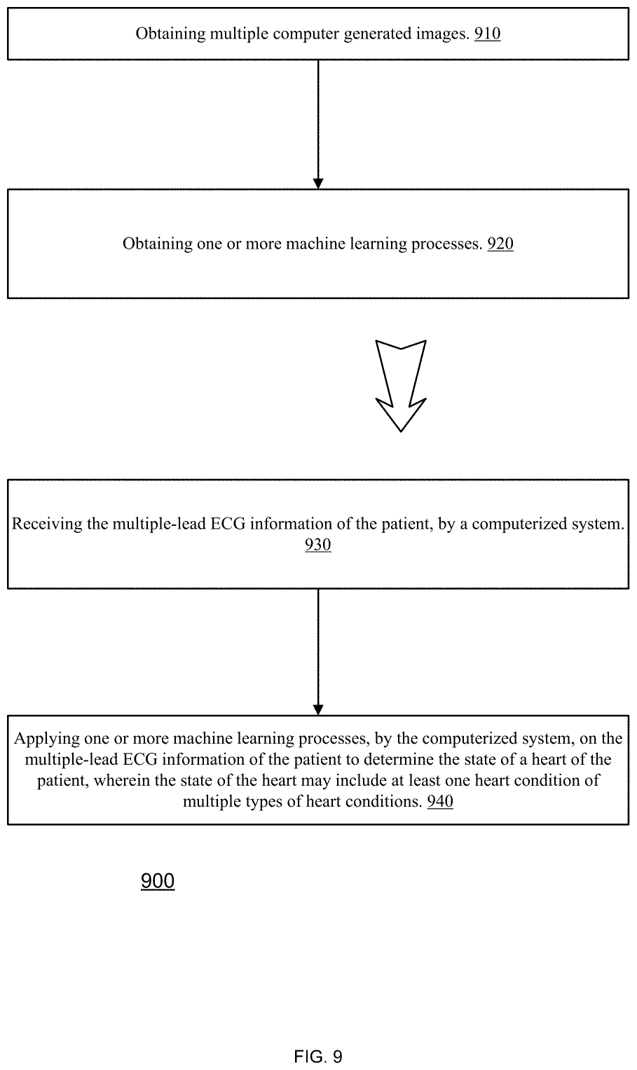

FIG. 9 illustrates an example of a method 900 for automatic determining of a state of a heart of a patient based on multiple-lead electrocardiogram (ECG) information of the patient.

Method 900 may start by one or more initialization steps.

The one or more initialization steps may include step 910 and/or step 920.

Step 910 may include obtaining multiple computer generated images.

Step 910 may include receiving computer generated images and/or generating computer generated images.

The multiple computer generated images represent images acquired by an image acquisition process of ECG plots. The image acquisition process may be an image acquisition process of an image sensor, of a smartphone, of a wearable device, and the like.

Step 910 may include receiving the ECG plots and/or generating the ECG plots based on digital multiple-lead ECG signals. The generating of the ECG plots may include selecting one or more ECG plots backgrounds.

Step 910 may include generating the computer generated images by applying a perspective transformation on the ECG plots.

Step 910 may include generating the computer generated images by applying an image acquisition function that mimics the image acquisition process.

Step 920 may include obtaining one or more machine learning processes.

Step 920 may include receiving a trained machine learning process and/or training a machine learning process. Step 920 may include receiving more than a single machine learning process and/or training more than a single machine learning process.

At least one machine learning process of the one or more machine learning processes was trained using a dataset that includes the multiple computer generated images. If there are, for example K machine learning processes, then the at least machine learning process may include one machine learning process till K machine learning processes, K being a positive integer.

The K machine learning processes may be multiple machine learning processes. Each machine learning process may be trained to classify a unique type of a heart condition of the multiple types of heart conditions.

For example a first machine learning process is trained to classify AF, a second is trained to classify I-AVB, a third is trained to classify LBBB, a fourth is trained to classify RBBB, a fifth is trained to classify PAC, a sixth is trained to classify PVC, a seventh is trained to classify STD, and an eighth is trained to classify STE.

There may be a subset of the eight mentioned above machine learning process, more than the eight mentioned above machine learning process, there may be machine learning process or processes configured to classify other types of heart conditions, and the like.

There may be provided a multiclass classifier for all K heart condition types or a classifier for subsets of K heart condition types. The subsets may include combinations of 2, 3, 4 heart conditions and/or of a combination of K/2, K/3, K/4 heart conditions.

The dataset may include digital multiple-lead ECG signals and at least one machine learning process may be trained based on the digital multiple-lead ECG signals.

The one or more initialization steps may be followed by step 930 of receiving the multiple-lead ECG information of the patient, by a computerized system.

Step 930 may be followed by step 940 of applying one or more machine learning processes, by the computerized system, on the multiple-lead ECG information of the patient to determine the state of a heart of the patient, wherein the state of the heart may include at least one heart condition of multiple types of heart conditions.

Step 940 may include or may be followed by responding to the state of the heart of the patient—for example storing the state, sending a notification to the patient and/or a medical staff member, send an alert to an emergency center (when the heart condition is critical), send instructions to the patient, update statistics of the heart condition, and the like, request another ECG measurement, and the like.

There may be multiple repetitions of steps 930 and 940 per a single iteration of the one or more initialization steps.

The computerized process is configured to apply the one or more machine learning processes.

The multiple-lead ECG information may be a 12-lead ECG information.

Step 930 may include receiving multiple-lead ECG information that is an image acquired by an image sensor.

Step 930 may include receiving multiple-lead ECG information that is multiple-lead ECG information comprises digital multiple-lead ECG signals. In this case the Step 930 may include or may be followed by converting the digital multiple-lead ECG signals to a computer generated image.

It should be noted that a machine learning process may be trained using digital multiple-lead ECG signals and that the received digital multiple-lead ECG signals may be sent to that machine learning process—without conversion to a computer generated image.

Any one of the one or more machine learning process may be implemented by one or more neural networks. Examples of such one or more neural networks may include CNNs, CNN-dig, CNN-ima or any other type of neural networks that are configured to execute method 900.

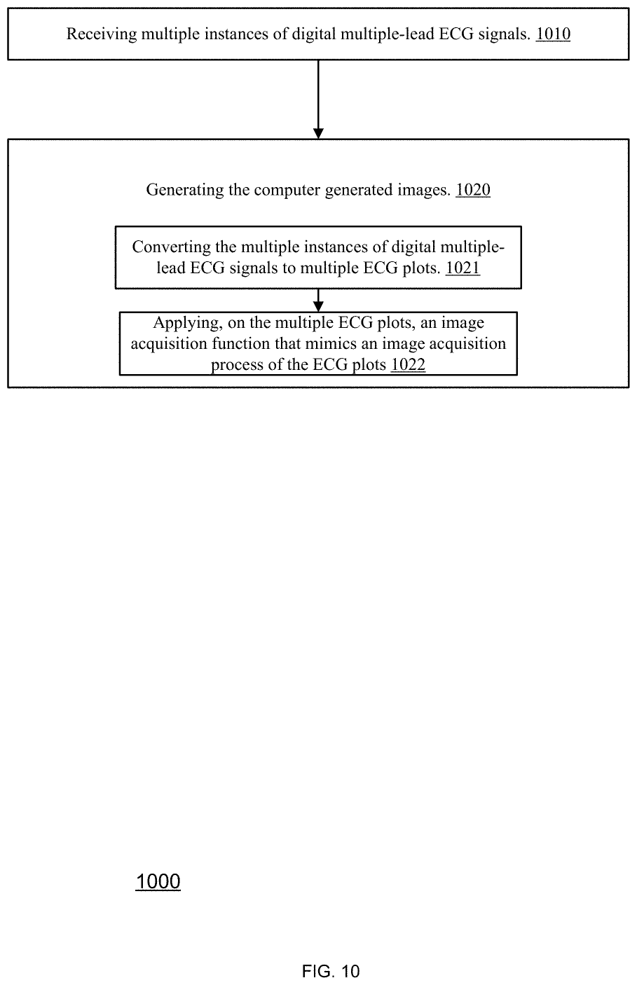

Method 900 may use for training and/or verifying a database of computer generated imaged. An example of a method for generating such a database is illustrated in FIG. 10.

FIG. 10 illustrates an example of method 1000 for generating a database of computer generated images that represent multiple-lead electrocardiogram (ECG) information.

Method 1000 may include step 1010 of receiving multiple instances of digital multiple-lead ECG signals.

Step 1010 may be followed by step 1020 of generating the computer generated images.

Step 1020 may include steps 1021 and 1022. Step 1021 may include converting the multiple instances of digital multiple-lead ECG signals to multiple ECG plots. Step 1022 may include applying, on the multiple ECG plots, an image acquisition function that mimics an image acquisition process of the ECG plots.

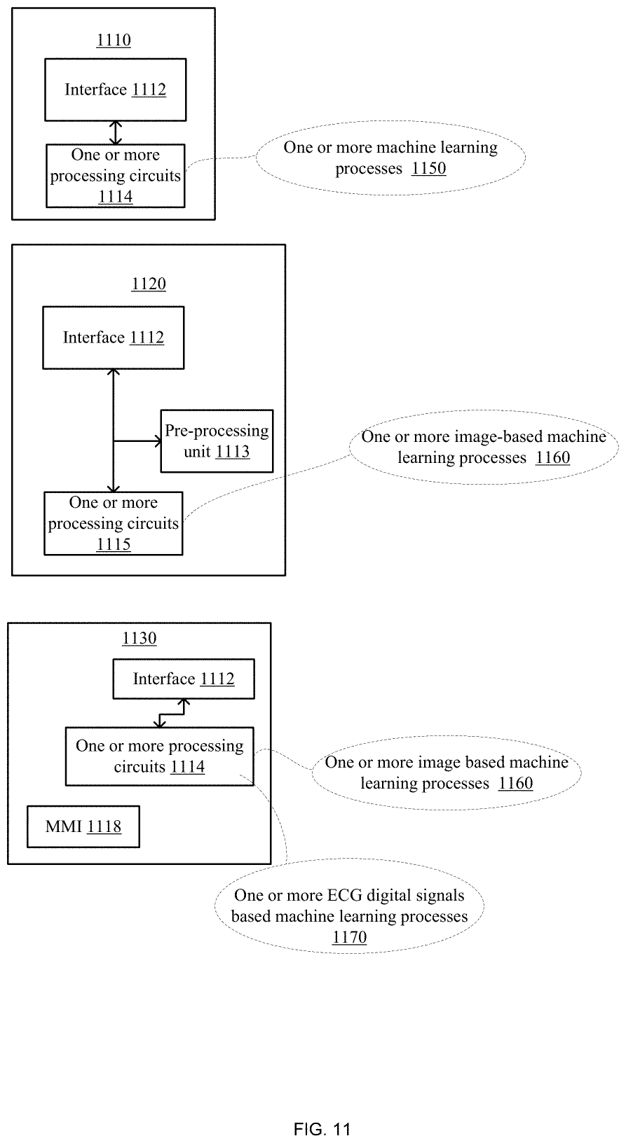

FIG. 11 illustrates examples of computerized systems 1110, 1120 and 1130 for automatic determining of a state of a heart of a patient based on multiple-lead electrocardiogram (ECG) information of the patient.

Computerized system 1110 includes an interface 1112 for receiving multiple-lead ECG information of the patient, and one or more processing circuits 1114 for applying one or more machine learning processes 1150 on the multiple-lead ECG information of the patient to determine the state of a heart of the patient. The state of the heart may include at least one heart condition of multiple types of heart conditions. Accordingly—the one or more machine learning processes may detect more than a single heat condition. The interface may be any communication unit and may use wireless or wired communication.

In one example, the one or more machine learning processes include (a) at least one image based machine learning process that is configured to determine the state of the heart based on multiple-lead ECG information that is an image of an ECG plot, and may also include (b) at least one ECG digital signals based machine learning process that is configured to determine the state of the heart based on multiple-lead ECG information that is digital multiple-lead ECG information.

Computerized system 1120 includes interface 1112, pre-processing unit 1113 and one or more processing circuits 1115 for applying one or more image based machine learning processes 1160 on the multiple-lead ECG information of the patient to determine the state of a heart of the patient. The state of the heart may include at least one heart condition of multiple types of heart conditions.

An image based machine learning process is a machine learning process that is configured to receive as input multiple-lead ECG information that is an image—for example an image of an ECG plot.

When receiving multiple-lead ECG information that is digital multiple-lead ECG information and not an acquired image, the pre-processor unit 1113 is configured to convert such information to a computer generated image—for example by executing step 1020 of method 1000. The pre-processor unit 1113 may include one or more processing circuits.

Computerized system 1130 includes interface 1112, and one or more processing circuits 1118.

The one or more processing circuits 1118 are configured to (a) determine whether the received multiple-lead ECG information is an image or is a digital multiple-lead ECG information, (b) supply (when the multiple-lead ECG information is an image) the image to one or more image based machine learning processes 1160, (c) supply (when the multiple-lead ECG information is digital multiple-lead ECG information) the digital multiple-lead ECG information to one or more ECG digital signals based machine learning processes 1170, and (d) apply the relevant one or more machine learning processes on the multiple-lead ECG information to determine the state of the heart.

Any of said computerized system s computerized system may include one or more man machine interfaces (for example a display and/or a microphone and/or any other man machine interfaces) and/or storage and/or memory and/or communication units other than the interface. For Example—see man machine interface (MMI) 1118 of computerized system 1130.

Any computerized system may be or may include one or more servers, one or more laptop computers, one or more integrated circuits, one or more mobile phones, one or more wearable computers, and the like.

A processing circuit may be implemented as a central processing unit (CPU), and/or one or more other integrated circuits such as application-specific integrated circuits (ASICs), field programmable gate arrays (FPGAs), full-custom integrated circuits, etc., or a combination of such integrated circuits.

Any reference to the term “comprising” or “having” should be applied mutatis mutandis to a reference to “consisting” and/or should be applied mutatis mutandis to “essentially consisting of”. For example—a method that comprises certain steps can include additional steps. A method that consists certain steps can be limited to the certain steps. A method that consists essentially of certain steps may include additional steps that do not materially affect the basic and novel characteristics of the method.

The invention may also be implemented in a computer program for running on a computer system, at least including code portions for performing steps of a method according to the invention when run on a programmable apparatus, such as a computer system or enabling a programmable apparatus to perform functions of a device or system according to the invention. The computer program may cause the storage system to allocate disk drives to disk drive groups.

A computer program is a list of instructions such as a particular application program and/or an operating system. The computer program may for instance include one or more of: a subroutine, a function, a procedure, an object method, an object implementation, an executable application, an applet, a servlet, a source code, an object code, a shared library/dynamic load library and/or other sequence of instructions designed for execution on a computer system.

The computer program may be stored internally on a computer program product such as non-transitory computer readable medium. All or some of the computer program may be provided on computer readable media permanently, removably or remotely coupled to an information processing system. The computer readable media may include, for example and without limitation, any number of the following: magnetic storage media including disk and tape storage media; optical storage media such as compact disk media (e.g., CD-ROM, CD-R, etc.) and digital video disk storage media; nonvolatile memory storage media including semiconductor-based memory units such as FLASH memory, EEPROM, EPROM, ROM; ferromagnetic digital memories; MRAM; volatile storage media including registers, buffers or caches, main memory, RAM, etc. A computer process typically includes an executing (running) program or portion of a program, current program values and state information, and the resources used by the operating system to manage the execution of the process. An operating system (OS) is the software that manages the sharing of the resources of a computer and provides programmers with an interface used to access those resources. An operating system processes system data and user input, and responds by allocating and managing tasks and internal system resources as a service to users and programs of the system. The computer system may for instance include at least one processing unit, associated memory and a number of input/output (I/O) devices. When executing the computer program, the computer system processes information according to the computer program and produces resultant output information via I/O devices.

In the foregoing specification, the invention has been described with reference to specific examples of embodiments of the invention. It will, however, be evident that various modifications and changes may be made therein without departing from the broader spirit and scope of the invention as set forth in the appended claims.

Moreover, the terms “front,” “back,” “top,” “bottom,” “over,” “under” and the like in the description and in the claims, if any, are used for descriptive purposes and not necessarily for describing permanent relative positions. It is understood that the terms so used are interchangeable under appropriate circumstances such that the embodiments of the invention described herein are, for example, capable of operation in other orientations than those illustrated or otherwise described herein.

Those skilled in the art will recognize that the boundaries between logic blocks are merely illustrative and that alternative embodiments may merge logic blocks or circuit elements or impose an alternate decomposition of functionality upon various logic blocks or circuit elements. Thus, it is to be understood that the architectures depicted herein are merely exemplary, and that in fact many other architectures may be implemented which achieve the same functionality.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Furthermore, those skilled in the art will recognize that boundaries between the above described operations merely illustrative. The multiple operations may be combined into a single operation, a single operation may be distributed in additional operations and operations may be executed at least partially overlapping in time. Moreover, alternative embodiments may include multiple instances of a particular operation, and the order of operations may be altered in various other embodiments.

Also for example, in one embodiment, the illustrated examples may be implemented as circuitry located on a single integrated circuit or within a same device.

Alternatively, the examples may be implemented as any number of separate integrated circuits or separate devices interconnected with each other in a suitable manner.

Also for example, the examples, or portions thereof, may implemented as soft or code representations of physical circuitry or of logical representations convertible into physical circuitry, such as in a hardware description language of any appropriate type.

Also, the invention is not limited to physical devices or units implemented in non-programmable hardware but can also be applied in programmable devices or units able to perform the desired device functions by operating in accordance with suitable program code, such as mainframes, minicomputers, servers, workstations, personal computers, notepads, personal digital assistants, electronic games, automotive and other embedded systems, cell phones and various other wireless devices, commonly denoted in this application as ‘computer systems’.

However, other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles.

Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim:

1. A method for automatic determining of a state of a heart of a patient based on multiple-lead electrocardiogram (ECG) information of the patient, the method comprises:
receiving the multiple-lead ECG information of the patient, by a computerized system; and
applying one or more machine learning processes on the multiple-lead ECG information of the patient to determine the state of a heart of the patient, wherein the state of the heart comprises at least one heart condition of multiple types of heart conditions;
wherein a machine learning process of the one or more machine learning processes was trained using a dataset that comprises multiple computer generated images, the multiple computer generated images represent images acquired by an image acquisition process of ECG plots, wherein the ECG plots are generated based on digital multiple-lead ECG signals.

2. The method according to claim 1 wherein the multiple computer generated images are generated by applying a perspective transformation on the ECG plots.

3. The method according to claim 1 wherein the multiple computer generated images are generated by applying an image acquisition function that mimics the image acquisition process.

4. The method according to claim 1 wherein the ECG plots are generated by selecting one or more ECG plots backgrounds.

5. The method according to claim 1 wherein the multiple-lead ECG information is a 12-lead ECG information.

6. The method according to claim 1 wherein the one or more machine learning processes are multiple machine learning processes, wherein each machine learning process is trained to classify a unique type of a heart condition of the multiple types of heart conditions.

7. The method according to claim 1 wherein the multiple-lead ECG information is an image acquired by an image sensor.

8. The method according to claim 1 wherein the multiple-lead ECG information comprises digital multiple-lead ECG signals, and wherein the method comprises converting the digital multiple-lead ECG signals to a computer generated image.

9. The method according to claim 1 wherein the dataset also comprised digital multiple-lead ECG signals.

10. The method according to claim 9 wherein the one or machine learning processes comprise a machine learning process trained based on the digital multiple-lead ECG signals.

11. A non-transitory computer readable medium for automatic determining of a state of a heart of a patient based on multiple-lead electrocardiogram (ECG) information of the patient, the non-transitory computer readable medium stored instructions that once executed by a computerized system cause the computerized system to:
receive the multiple-lead ECG information of the patient, by a computerized system; and
apply one or more machine learning processes on the multiple-lead ECG information of the patient to determine the state of a heart of the patient, wherein the state of the heart comprises at least one heart condition of multiple types of heart conditions;
wherein a machine learning process of the one or more machine learning processes was trained using a dataset that comprises multiple computer generated images, the multiple computer generated images represent images acquired by an image acquisition process of ECG plots, wherein the ECG plots are generated based on digital multiple-lead ECG signals.

12. The non-transitory computer readable medium according to claim 11 wherein the multiple computer generated images are generated by applying a perspective transformation on the ECG plots.

13. The non-transitory computer readable medium according to claim 11 wherein the multiple computer generated images are generated by applying an image acquisition function that mimics the image acquisition process.

14. The non-transitory computer readable medium according to claim 11 wherein the ECG plots are generated by selecting one or more ECG plots backgrounds.

15. The non-transitory computer readable medium according to claim 11 wherein the multiple-lead ECG information is a 12-lead ECG information.

16. The non-transitory computer readable medium according to claim 11 wherein the one or more machine learning processes are multiple machine learning processes, wherein each machine learning process is trained to classify a unique type of a heart condition of the multiple types of heart conditions.

17. The non-transitory computer readable medium according to claim 11 wherein the multiple-lead ECG information is an image acquired by an image sensor.

18. The non-transitory computer readable medium according to claim 11 wherein the multiple-lead ECG information comprises digital multiple-lead ECG signals, and wherein the non-transitory computer readable medium stored instructions for converting the digital multiple-lead ECG signals to a computer generated image.

19. The non-transitory computer readable medium according to claim 11 wherein the dataset also comprised digital multiple-lead ECG signals.

20. The non-transitory computer readable medium according to claim 19 wherein the one or machine learning processes comprise a machine learning process trained based on the digital multiple-lead ECG signals.

21. A method for generating a database of computer generated images that represent multiple-lead electrocardiogram (ECG) information, the method comprising:
receiving multiple instances of digital multiple-lead ECG signals; and
generating the computer generated images by:
converting the multiple instances of digital multiple-lead ECG signals to multiple ECG plots; and
applying, on the multiple ECG plots, an image acquisition function that mimics an image acquisition process of the ECG plots.
